(19) United States
(12) Patent Application Publication
(10) Pub. No.: US 2006/0062749 A1
(43) Pub. Date: Mar. 23, 2006

(54) personal care products incorporating cellulosic fatty acid esters

(76) Inventors: Michael Charles Shelton, Kingsport, TN (US); Eric Eugene Ellery, Kingsport, TN (US); Chung-Ming Kuo, Kingsport, TN (US); Debra Tindall, Kingsport, TN (US); Terry Ann Oldfield, Kingsport, TN (US)

Correspondence Address: Michael J. Blake;Eastman Chemical Company, P.O. Box 511, Kingsport, TN, 37662-5075, US

(21) Appl. No.: 11/226,802

(22) Filed: Sep. 14, 2005

Related U.S. Application Data
(60) Provisional application No. 60/610,367, filed on Sep. 16, 2004.

(51) Int. Cl. A61K 8/73

(52) U.S. Cl. 424/70.13

(57) ABSTRACT

A personal care product composition containing a long chain fatty acid cellulose ester (LCCE) having a degree of substitution greater than about 1.0 of an ester substituent or residue from fatty acids having from 6 to 18 carbon atoms wherein the LCCE is soluble in at least one cosmetically acceptable solvent selected from the group consisting of hydrocarbons, alkyl esters, fats and oils, fatty acids, fatty alcohols, and silicone oils.

### CROSS REFERENCE TO RELATED APPLICATIONS

Benefit is claimed to the earlier filed application having U.S. Ser. No. 60/610,367 filed Sep. 16, 2004, the entire disclosure of which is incorporated herein by reference.

### FIELD OF THE INVENTION

The present invention relates to personal care products that include a fatty acid cellulose ester. More particularly, the present invention relates to personal care products that include a fatty acid cellulose ester having a degree of substitution (DS) of greater than about 1.0 of an ester substituent having from 6 to 18 carbon atoms.

### BACKGROUND OF THE INVENTION

Fatty acid esters of cellulose and particularly long chain esters of cellulose chemically have long chain saturated fatty acid moieties esterified onto the hydroxyls of the glucose moieties in cellulose. Processes and procedures for synthesis of such long chain esters of cellulose are well known in the art. For example, Malm, C. J.; Mench, J. W.; Kendall, D. L.; Hiatt, G. D. “Aliphatic Acid Esters of Cellulose: Preparation by Acid Chloride-Pyridine Procedure,” Ind. Eng. Chem. 1951, 43, 684, describes the preparation of a series of cellulose esters from acetate through palmitate by the acid chloride-pyridine procedure, in order to maintain the same degree of polymerization of the starting cellulose acetate. Kwatra, H. S.; Caruthers, J. M; and Tao, B. Y., “Synthesis of Long Chain Fatty Acids Esterified onto Cellulose via the Vacuum-Acid Chloride Process”, Ind. Eng. Chem. 1992, 31, 2647-2651 describes a process wherein palmitoyl fatty acid ester of cellulose was prepared by using vacuum to remove hydrogen chloride produced during the condensation reaction thereby eliminating solvents from the reaction.

Direct synthesis of partially substituted cellulose esters has been taught previously by acylation of cellulose in solution as shown in U.S. Pat. No. 2,976,277. If cellulose is first dissolved in a mixture of lithium chloride and an amide solvent (either 1-methyl-2-pyrrolidinone (NMP) or N,N-dimethylacetamide (DMAC)), it can then be acylated with a carboxylic anhydride in the presence or absence of a catalyst to afford a partially or fully substituted cellulose ester depending only on the equivalents of anhydride added. Esters of cellulose with long-chain carboxylic acids have been made in this way.

U.S. Pat. No. 5,929,229 to Edgar et al. describes a direct heterogenous process for preparing cellulose esters of less than full substitution by the reaction of cellulose in a carboxamide diluent or a urea-based diluent with an acylating agent such as carboxylic acid anhydride using a titanium-containing catalyst.

Additionally, U.S. Pat. No. 6,160,111 to Edgar et al. describes a process for direct heterogenous process for preparing cellulose esters of less than full substitution by the reaction of cellulose in a carboxamide diluent or a urea-based diluent with an acylating agent such as carboxylic acid anhydride using an insoluble sulfonic acid resin catalyst.

Commercially, partially substituted cellulose esters have been utilized in such applications as coatings, plastics, fibers, and film manufacture. In the area of coatings, the greater solubility and hydroxyl group content are valued. In the area of personal care, cellulose esters having substituents of from 2 to 4 carbon atoms only, such as cellulose acetate propionate and cellulose acetate butyrate, have been used as the primary or secondary film-former in finger nail coatings. Additionally, International publication WO 2005/013926 discloses using substituted cellulose esters, and particularly cellulose esters that are liposoluble wherein the free hydroxyl moieties are replaced by hydrophobic groups having one or more substituents from 4 to 50 carbon atoms. The publication defines “liposoluble” as having a solubility of at least 1 weight % in the principal oil of the liquid fat phase at ambient temperature and pressure. However, it has unexpectedly been discovered that long chain fatty acid cellulose esters disclosed in publication WO 2005/013926 are not soluble in solvents or organic carriers commonly used in cosmetic and personal care applications.

Cosmetics and personal care products that are oil-based or have an oil phase have limited durability on the lips or skin. For example color cosmetics wear off after a limited amount of time when subjected to forces of smudging or smearing, especially when accompanied by perspiration. Skin care products, in the case of sunscreens for example, rub off when contacted by clothing or rinse off while swimming. Also, compositions that contain slowly penetrating active ingredients need to be left on the skin for a long period of time to allow the active ingredient as much time as possible to absorb into the skin. To improve durability and water resistance of such compositions, it is desirable that they contain an oil-soluble film-former.

Compositions such as color cosmetics, deodorants, skin care creams and lotions, and hair preparations need to be thickened so that they can be applied in the form of a stick or can be poured into and contained in the hand and applied with the fingers. Thickening is also beneficial so that compositions stay where they are placed rather than running or dripping away from the intended substrate. It is desirable for a thickened composition to be shear thinning to provide ease of spreading or when sprayed to provide a fine droplets and even distribution.

Accordingly, there is a need for a personal care item that has good performance, long lasting, suitable texture and is easy to apply.

### SUMMARY OF THE INVENTION

Unexpectedly, it has been discovered that a long chain fatty acid cellulose ester (LCCE) having a degree of substitution (DS) on the cellulose moiety of greater than about 1.0 of an ester substituent having from 6 to 18 carbon atoms is soluble in at least one cosmetically acceptable solvent selected from hydrocarbons, alkyl esters, fats and oils, fatty acids, fatty alcohols, and silicone oils.

### DETAILED DESCRIPTION OF THE INVENTION

The personal care items of the present invention include deodorants, antiperspirants, combination antiperspirant deodorants, shaving products, skin lotions, moisturizers, toners, bath products, cleansing products, hair care products, shampoos, conditioners, mousses, styling gels, hair sprays, hair dyes, hair coloring products, hair bleaches, hair waving products, hair straighteners, manicure products, nail polish, nail polish remover, nail creams, nail lotions, cuticle softeners, protective creams, sunscreen products, insect repellent, anti-aging products, color cosmetics, lipsticks, foundations, face powders, eye liners, eye shadows, blushes, makeup, mascara, personal care formulations where cellulosic components have been conventionally added, and drug delivery systems for topical application of medicinal compositions that are to be applied to the skin. In accordance with the present invention, the personal care product includes a long chain fatty acid cellulose ester (LCCE) having a degree of substitution (DS) on the cellulose moiety of greater than about 1.0 of an ester substituent or residue from fatty acids having from 9 to 18 carbon atoms. Preferably, the long chain fatty acid cellulose ester has a degree of substitution greater than about 1.5, more preferably greater than about 2.0, and most preferably greater than about 2.5 of an ester substituent or residue from fatty acids having from 9 to 18 carbon atoms. Surprisingly, the LCCE is soluble in at least one cosmetically acceptable solvent selected from hydrocarbons, alkyl esters, fats and oils, fatty acids, fatty alcohols, and silicone oils. In a particularly preferred embodiment, the cellulose moiety has an acetyl degree of substitution of less than 0.5 and preferably less than about 0.3.

Typically, the personal care product includes from about 0.1 to about 10 weight % of the LCCE based on the total weight of the constituents in the product composition. Desirably, the personal care product includes from about 0.5 to about 8 weight % of the LCCE, and more preferably from about 0.5 to about 5 weight %.

Generally, the cellulose fatty acid esters can be prepared by a variety of processes, such as: acid-catalyzed transesterification of commercial cellulose esters with fatty acids; base-catalyzed transesterification of commercial cellulose esters with fatty acids; acid-catalyzed direct esterification of cellulose using fatty acid anhydrides; acid-catalyzed direct esterification of cellulose using fatty acid chlorides, and acid-catalyzed direct esterification of cellulose using fatty acid mixed anhydrides. The cellulose used to prepare the long chain fatty acid cellulose esters can come from a variety of sources. Cellulose sources useful in preparing the LCCE include hardwood pulp, softwood pulp, cotton linters, bacterial cellulose, and regenerated cellulose. Processes and procedures used to prepare the LCCEs are described in greater detain in Gedon, S.; Fengl, R. “Cellulose Esters,” Kirk-Othmer Encylopedia of Chemical Technology, 4th Ed., vol. 5, John Wiley & Sons, New York, 1993, pp. 496-529, (describes the preparation of cellulose esters in sufficient detail that those skilled in the art can prepare starting materials used in this invention) as well as the literature and patents presented above, the entire disclosures of each are incorporated herein by reference.

Desirably, the LCCE have a degree of substitution containing C₆-C₁₈ fatty acid residual content greater than about 1.0. As used herein, the term “degree of substitution”, “DS” or “DS/AGU” refers to the average number of acyl substituents per anyhydroglucose ring of the cellulose polymer where the theoretical maximum DS is 3. The LCCEs useful in the present invention have a total DS/AGU greater than about 1.0, preferably greater than about 1.5, more preferably greater than about 2.0. For the cellulose esters of this invention, DS or DS/AGU may be determined using any method known in the art. For example, using proton NMR. DS can be determined by ¹H NMR in d-6 dimethylsulfoxide (DMSO) or tetrahydrofuran (THF) containing several drops of trifluoroacetic acid (to shift any hydroxyl protons downfield), or in tetrachloroethane containing several drops of trifluoroacetyl isocynate, or by hydrolysis of a sample of the cellulose ester followed by quantification of liberated carboxylic acids by gas chromatography.

The LCCE's of the invention typically have a weight average molecular weight (M_{W}) as measured by gel permeation chromatography in THF of about 20,000 to about 8,000,000.

Preferred cellulose esters useful in the personal care products of the present invention include cellulose isostearate, cellulose palmitate, cellulose nonanoate, cellulose hexanoate, cellulose acetate hexanoate, cellulose acetate nonanoate, cellulose acetate laurate, cellulose acetate stearate, cellulose hexanoate propionate, and cellulose nonanoate propionate.

More preferred LCCEs suitable for use in the present personal care products are those that are soluble in solvents or organic carriers commonly used in cosmetic and personal care applications, such as cellulose isostearate, cellulose nonanoate, cellulose acetate nonanoate, and mixtures thereof. As used herein, the LCCE is “soluble” if the LCCE is completely dissolved at a concentration of 1 weight % or greater, based on the total weight of the composition, in the oil phase solvent or carrier and the mixture forms a clear, homogeneous liquid, gel, or waxy solid after it has cooled and remained at room temperature (25° C.) for at least 24 hours. The solution can be made by heating the components to a temperature up to about 90° C. with stirring or other agitation. Solvents or organic carriers commonly used in cosmetic and personal care applications include, but are not limited to hydrocarbons, alkyl esters, fats and oils, fatty acids, fatty alcohols, and silicone oils.

Typical hydrocarbons include isoparaffins, hydrogenated polyisobutene, isododecane, isoeicosane, isohexadecane, isopentane, microcrystalline wax, mineral oil, mineral spirits, paraffin, petrolatum, squalene, polyethylene, natural waxes such as carnauba wax and candelilla wax and mixtures thereof. Examples of further hydrocarbons are set forth on pages 2136 and 2137 of the CTFA International Cosmetic Ingredient Handbook, Tenth Edition, 2004, which is hereby incorporated by reference.

Suitable alkyl esters are those in which the inventive cellulose ester is soluble, preferably where the alkyl portion has at least eight carbon atoms. These include alkyl acetates, alkyl behenates, alkyl lactates, alkyl benzoates, alkyl salicylates, typical alkyl fatty acid esters such as alkyl stearates, alkyl palmitates, alkyl myristates, and alkyl laurates, and mixtures thereof.

Typical fats and oils, further defined as glyceryl esters of fatty acids (triglycerides), also includes synthetically prepared esters of glycerin and fatty acids. Examples include soybean oil, corn oil, canola oil, olive oil, sunflower oil, triolein, tristearin, caprylic/capric triglyceride, and mixtures thereof.

Typical fatty acids are obtained by hydrolysis of animal or vegetable fats and oils. Examples include valeric acid, heptylic acid, caprylic acid, lauric acid, myristic acid, and palmitic acid, behenic acid, capric acid, caproic acid, coconut acid, oleic acid, linoleic acid, palmitic acid, isopalmitic acid, stearic acid, isostearic acid, and mixtures thereof.

Typical fatty alcohols are those derived by reducing the fatty acid to the hydroxyl function. Examples of suitable fatty alcohols are C₉-C₃₀ alcohols, branched and straight chain. These include lauryl alcohol, isolauryl alcohol, cetyl alcohol, isocetyl alcohol, stearyl alcohol, isostearyl alcohol, octyldodecanol, octyl tetradecanol, dodecyl hexadecanol, hexadecyl eicosanol, and mixtures thereof.

Silcone oils include those compatible with an oil-based solution of the cellulose ester, including volatile and non-volatile silicone oils, linear and cyclic. Examples include dimethicone, hexadecyl methicone, stearyl dimethicone, cyclomethicone, cyclopentasiloxane, phenyl trimethicone, and mixtures thereof.

The cellulose fatty acid esters are soluble in liquid carriers typically used in oil-based cosmetic products or as part of the oil-phase in cosmetic/personal care emulsions. Cosmetic/personal care emulsions include oil-in-water, water-in-oil, as well as multiple emulsions, such as for example oil-in-water-in-oil and water-in-oil-in-water emulsions. Such emulsions typically contain emulsifying agents or surfactants to allow the oil phase and water phase to mix in such a way that one or the other forms a continuous phase, while the other forms a discontinuous phase that is typically suspended in the form of micelles in the continuous phase. In such an emulsion, the oil phase can contain those ingredients described above as typical organic carriers in oil-based products. The water or aqueous phase may contain any ingredients that are compatible and/or soluble in water. For skin care products, these typically include humectants such as glycols, sugars, and the like. Examples of suitable glycols include propylene glycol, polyethylene glycols, polypropylene glycols, and glycerin. Examples of sugars include glucose, fructose, inositol, and sucrose. Other water-soluble ingredients include gellants such as water-soluble or swellable gums, and water soluble polymers, including polymers of acrylic acid and esters thereof.

Other suitable personal care ingredients include, for example, cleansing agents, emollients, moisturizers, pigments, including pearlescent pigments, colorants, fragrances, biocides, preservatives, antioxidants, antiperspirant agents, oral care agents, exfoliants, hormones, enzymes, medicinal compounds, vitamins, ultraviolet light absorbers, dihydroxyacetone, skin bleaching agents, antiacne agents, botanical extracts, silicone oils, organic oils, waxes, adhesion promoters, plasticizers, film formers, including hair fixatives, thickening agents, fillers and binders, alcohol and other organic solvents, and propellants.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### COMPARATIVE EXAMPLE 1

Cellulose acetate nonanoate was prepared from cellulose acetate by the pyridine-acid chloride process, a process similar to that described by C. J. Malm, et al, Industrial and Engineering Chemistry, vol 43, pages 684-688, 1951.

The following reagents were added, in the following order, to a one liter, three neck, round bottom flask, equipped with a stirrer and cold water condenser/distillation column, and placed in a silicone oil bath: 500 mL of N-methyl pyrrolidone—(C₅H₉NO), (NMP); 17 mL of pyridine—(C₅H₅N); 30 grams of oven dried, cellulose acetate (cellulose acetate with an apparent acetyl between 31.0 and 33.0 weight %, an intrinsic viscosity in pyridine of approximately 0.88 dL/g and a weight average molecular weight of approximately 47,500 Daltons, measured by size exclusion chromatography in N-methyl pyrrolidone). The cellulose acetate was prepared in a manner similar to that described in Gedon, S.; Fengl, R. “Cellulose Esters,” Kirk-Othmer Encylopedia of Chemical Technology, 4th Ed., vol. 5, John Wiley & Sons, New York, 1993, pp. 496-529). This mixture was stirred at room temperature until the cellulose acetate was dissolved. To this mixture, 27 mL of nonanoyl chloride (C₉H₁₇ClO) was added drop wise over approximately 30 minutes with constant stirring. After the addition of the nonanoyl chloride, the entire mixture was warmed to 90-91° C. and stirred at this temperature for 24 hours. After 24 hours, 35 mL of deionized water was added to the reaction mixture to assure the decomposition of any remaining nonanoyl chloride. The resulting cellulose ester product was precipitated by stirring the reaction mixture into methanol. After several changes of methanol to wash the product free of solvents, the product was washed in a tap wash bag with deionized water over night. The product was dried in a vacuum oven under a nitrogen purge for 24 hours at 50-80° C. The resulting dry product was analyzed by NMR and found to contain DS acetyl of 1.818 and a DS nonanoyl of 0.902. The weight-average molecular weight (Mw) was determined to be 1.09×10⁵ Daltons using gel permeation chromatography in tetrahydrofuran. The product was acetone soluble and not soluble in isohexadecane, (Creasil IH™), or isododecane, (Creasil ID™). (Creasil IH™ and Creasil ID™ are trade names of Optima Specialty Chemical LLC).

### COMPARATIVE EXAMPLE 2

Cellulose acetate nonanoate was prepared from cellulose acetate by the pyridine-acid chloride process.

The following reagents were added, in the following order, to a one liter, three neck, round bottom flask, equipped with a stirrer and cold water condenser/distillation column, and placed in a silicone oil bath: 292 mL of N-methyl pyrrolidone; 28 mL of pyridine; 30 grams of oven dried cellulose acetate (cellulose acetate with an apparent acetyl between 17.0 and 19.0 weight % and a weight average molecular weight, measured by size exclusion chromatography in N-methyl pyrrolidone, of approximately 20,000 Daltons, prepared in a manner similar to that described in Comparative Example 1 above. This mixture was stirred at room temperature until the cellulose acetate was dissolved. After the cellulose ester dissolved, 18 mL of solvent was distilled off to assure that any remaining water was removed from the reaction. To this mixture, 73 mL of nonanoyl chloride (C₉H₁₇ClO) was added drop wise over approximately 30 minutes with constant stirring. After the addition of the nonanoyl chloride the entire mixture was warmed to 95° C. and stirred at this temperature for 24 hours. After 24 hours, 35 mL of deionized water was added to the reaction mixture to assure the decomposition of any remaining nonanoyl chloride. The resulting product cellulose ester was precipitated by stirring the reaction mixture into 50/50 deionized water/methanol mixture. After several changes of methanol to wash the product free of solvent, the product was washed in a tap wash bag with deionized water over night. The product was dissolved in acetone, precipitated and washed by the above procedure to produce a small particle precipitate. The product was dried in a vacuum oven under a nitrogen purge for 24 hours at 50-80° C. The resulting dry product was analyzed by NMR and found to contain DS acetyl of 0.76 and a DS nonanoyl of 2.44. The total DS is greater than 3.0, possibly because the product may contain free acid impurities. The weight-average molecular weight (Mw) was measured by gel permeation chromatography in tetrahydrofuran and found to be 6.5×104 Daltons. The product was acetone soluble, toluene soluble and only swelled in isohexadecane or isododecane.

### COMPARATIVE EXAMPLE 3

Cellulose acetate butyrate nonanoate was prepared from cellulose acetate butyrate by the pyridine-acid chloride process.

The following reagents were added, in the following order, to a one liter, three neck, round bottom flask, equipped with a stirrer and cold water condenser/distillation column, and placed in a silicone oil bath: 438 mL of N-methyl pyrrolidone; 46 mL of pyridine; 30 grams of oven dried, cellulose acetate butyrate (CAB), having an acetyl content of approximately 4.01 weight %, a butyryl content of approximately 28.37 weight % and a hydroxyl content of approximately 1.30 weight %, a weight average molecular weight of approximately 40,600 Daltons, measured by size exclusion chromatography in N-methyl pyrrolidone. (The CAB was prepared in a manner similar to that described in Gedon, S.; Fengl, R. “Cellulose Esters,” Kirk-Othmer Encylopedia of Chemical Technology, 4th Ed., vol. 5, John Wiley & Sons, New York, 1993, pp. 496-529). The mixture was stirred at room temperature until the CAB was dissolved. After dissolution of the CAB, 30 mL of solvent was distilled off the reaction mixture. To this mixture, 81 mL of nonanoyl chloride (C₉H₁₇ClO) was added drop wise over approximately 45 minutes with constant stirring. After the addition of the nonanoyl chloride the entire mixture was warmed to 95° C. and stirred at this temperature for 24 hours. After 24 hours, 35 mL of deionized water was added to the reaction mixture to assure the decomposition of any remaining nonanoyl chloride. The reaction product was a gelled mass in the reaction flask. The resulting cellulose ester product was precipitated by stirring the reaction mixture into 50/50 deionized water/methanol mixture and made a soft precipitate that wanted to reform into a mass if left still in the precipitation liquids. After three redisolutions and re-precipitations and washings the product produced a particle precipitate. After several changes of methanol to wash the product free of solvents, the product was washed in a tap wash bag with deionized water over night. The product was dried in a vacuum oven under a nitrogen purge for 24 hours at 50° C. The resulting dry product was difficult to analyze by NMR due to interference from the butyryl signal in the nonanoyl range. The weight-average molecular weight (Mw) was measured by gel permeation chromatography in tetrahydrofuran and found to be 1.4×10⁵ Daltons. The product was acetone soluble, toluene soluble and not soluble in isohexadecane or isododecane.

### COMPARATIVE EXAMPLE 4

Cellulose acetate laurate was prepared from cellulose acetate by the pyridine-acid chloride process.

The following reagents were added, in the following order, to a one liter, three neck, round bottom flask, equipped with a stirrer and cold water condenser/distillation column, and placed in a silicone oil bath: 324 mL of pyridine and 30 grams of oven dried cellulose acetate, similar to that described in Comparative Example 1. This mixture was stirred at room temperature until the cellulose acetate dissolved. After dissolution of the cellulose acetate, 20 mL of solvent was distilled off the reaction mixture. To this mixture, 43 mL of lauroyl chloride (C₁₆H₃₁ClO) was added drop wise over approximately 30 minutes with constant stirring. After the addition of the lauroyl chloride the entire mixture was warmed to 90-91° C. and stirred at this temperature for 24 hours. After 24 hours, 25 mL of deionized water was added to the reaction mixture to assure the decomposition of any remaining lauroyl chloride. The resulting cellulose ester product was precipitated by stirring the reaction mixture into deionized water and after several changes of methanol to wash the product free of solvent, the product was washed in a tap wash bag with deionized water over night. The product was dried in a vacuum oven under a nitrogen purge for 24 hours at 80° C. The resulting dry product was analyzed by NMR and found to contain DS acetyl of 1.92 and a DS laurate of 1.42. The total DS is greater than 3.0, possibly because the product may contain free acid impurities. The weight-average molecular weight (Mw) was measured by gel permeation chromatography in tetrahydrofuran and found to be 9.2×10⁴ Daltons. The product was soluble in acetone, dimethyl chloride and n-propyl acetate, partially soluble in toluene, and not soluble in isohexadecane or isododecane, acetic acid or isopropanol.

### COMPARATIVE EXAMPLE 5

Cellulose acetate palmitate was prepared from cellulose acetate by the pyridine-acid chloride process.

The following reagents were added, in the following order, to a one liter, three neck, round bottom flask, equipped with a stirrer and cold water condenser/distillation column, and placed in a silicone oil bath: 307 mL of pyridine; 21 mL of N-methyl pyrrolidone; and 30 grams of oven dried cellulose acetate similar to that described in Comparative Example 1. This mixture was stirred at room temperature until the cellulose acetate dissolved. After dissolution of the cellulose acetate, 31 mL of solvent was distilled off the reaction mixture. To this mixture, 56 mL of palmitoyl chloride (C₁₂H₂₃ClO) was added drop wise over approximately 30 minutes with constant stirring. After the addition of the palmitoyl chloride, the entire mixture was warmed to 95° C. and stirred at this temperature for 24 hours. After 24 hours, 25 mL of deionized water was added to the reaction mixture to assure the decomposition of any remaining palmitoyl chloride. The resulting product cellulose ester was precipitated by stirring the reaction mixture into deionized water and reprecipitated from an acetone solution. After several methanol washes to wash the product free of solvent, the product was washed in a tap wash bag with deionized water over night. The product was Soxhlet extracted for 12 hours with methanol and was dried in a vacuum oven under a nitrogen purge for 24 hours at 80° C. The weight-average molecular weight (Mw) was measured by gel permeation chromatography in tetrahydrofuran and found to be 1.10×10⁵ daltons. The product from this example was only slightly swelled in isohexadecane or isododecane, acetic acid or isopropanol.

### COMPARATIVE EXAMPLE 6

Cellulose stearate was prepared from a soft wood pulp with an α-cellulose content greater than 94 weight %, (available from Rayonier) using the trifloroacetic anhydride, stearic acid method as described in Morooka, T., Norimot, M., Yamada, T., Jour. Applied Polymer Science, 1984, 29, 3981).

The following reagents were added, in the following order, to a one liter, three neck, round bottom flask, equipped with a stirrer and cold water condenser/distillation column, and placed in a silicone oil bath: 78.4 mL (117 g) of trifloroacetic anhydride and 194 grams of stearic acid. The mixture was stirred at 50° C. until the stearic acid dissolved and a mixed anhydride solution formed. To this solution, 10 grams of the wood pulp cellulose were added with stirring and the reaction mixture was held at 50° C. overnight with constant stirring. Approximately 200 mL of toluene was added to dilute the mixture. One half of this diluted mixture was precipitated into methanol.

Sulfuric acid (0.1 gram) was added to the other half of the mixture. This mixture was stirred at 50° C. for approximately 3 hours. The sulfuric acid was neutralized with magnesium acetate tetrahydrate. This reaction mixture was then precipitated into methanol.

Both parts of this experiment were then washed first in deionized water and then in methanol. The product ester from both halves had a weight-average molecular weight of about 3.5×10⁶ as measured by gel permeation chromatography. The product ester from both halves formed a hazy gel in isododecane and a hazy dispersion in isohexadecane.

### EXAMPLE 7

The following reagents were added, in the following order, to a one liter, three neck, round bottom flask, equipped with a stirrer and cold water condenser/distillation column, and placed in a silicone oil bath: 34.3 mL (51 g) of trifloroacetic anhydride and 93 grams of stearic acid were stirred at 50° C. until the stearic acid dissolved and a mixed anhydride solution was formed. To this solution, 10 grams of oven dried cellulose acetate, similar to that described in Comparative Example 2, was added. Continuously stirring, the reaction mixture was held at 50° C. and allowed to react for 5 hours. The resulting product was isolated by precipitation into methanol (5× vol./vol.). The precipitated cellulose acetate stearate product was washed with methanol, then washed with deionized water then again with methanol. Product was dried in a vacuum oven with a nitrogen purge at 35° C. The product had a DS stearate of 2.95 and a DS acetate of 0.82 and was soluble in both isohexadecane and isododecane. The total DS is greater than 3.0, possibly because the product may contain free acid impurities. The product weight-average molecular weight (Mw) was measured by gel permeation chromatography in tetrahydrofuran and found to be 6.5×10⁴ daltons.

### EXAMPLE 8

Cellulose nonanoate was prepared from wood pulp using a trifluoroacetic anhydride, nonanoic acid method.

The following reagents were added, in the following order, to a 500-mL, three neck, round bottom flask, equipped with a stirrer and cold water condenser and placed in a silicone oil bath: 44 grams of nonanoic acid and 49 grams of trifluoroacetic anhydride. The mixture was heated at 50° C. for 1 hour to form a mixed anhydride. To this solution, 5 grams of a soft wood pulp with an a-cellulose content greater than 95 weight %, was added with stirring. The reaction mixture was held at 50° C. overnight with constant stirring. This reaction mixture was then precipitated into methanol, washed first in deionized water and then in methanol. The precipitated and washed product was dried at 50° C. under vacuum. The resulting cellulose nonanoate ester had a DS nonanoate of 3.0 and was soluble in isododecane and isohexadecane. The product weight-average molecular weight (Mw) was measured by gel permeation chromatography in tetrahydrofuran and found to be 6.3×10⁵ daltons.

### COMPARATIVE EXAMPLE 9

Cellulose acetate nonanoate was prepared from cellulose acetate using trifluoroacetic anhydride nonanoic acid method.

The following reagents were added, in the following order, to a 500-mL, three neck, round bottom flask, equipped with a stirrer and cold water condenser and placed in a silicone oil bath: 44 grams of nonanoic acid and 49 grams of trifluoroacetic anhydride. The mixture was heated at 50° C. for 1 hour to form a mixed anhydride. To this solution, 5 grams of cellulose acetate, similar to that described in Comparative Example 2, was added with stirring and the reaction mixture was held at 50° C. overnight with constant stirring. This reaction mixture was then precipitated into methanol, washed first in deionized water and then in methanol. The precipitated and washed product was dried at 50° C. under vacuum. The resulting cellulose acetate nonanoate ester had a DS nonanoate of 2.48 and a DS acetate of 1.02 and was insoluble in isododecane and isohexadecane. The total DS is greater than 3.0, possibly because the product may contain free acid impurities. The product weight-average molecular weight (Mw) was measured by gel permeation chromatography in tetrahydrofuran and found to be 3.9×10⁴ daltons.

### EXAMPLES 10-23

Cellulose esters and the mixed cellulose acetate esters of long chain saturated fatty acids prepared from cotton linters using the trifluoroacetic anhydride carboxylic acid method.

Cellulose esters and the mixed cellulose acetate esters of long chain saturated fatty acids were prepared using the quantities of reagents shown in Table 1 below. A 500 mL, three neck, round bottom flask was equipped with a stirrer and cold water cooled vacuum distillation apparatus and placed in a silicone oil bath. The appropriate amount and type of carboxylic acid(s) for each of the example batches was added to the flask. Then the specified amount of trifluoroacetic anhydride (TFAA) was added drop wise with stirring. While continuously stirring, the mixture was heated to 50° C. and held at this temperature for 30 to 45 minutes to allow formation of the mixed anhydride(s). To this solution, the specified amount of cotton linter cellulose (high purity dissolving-grade cellulose isolated from commercial cotton bolls) was added and the reaction mixture was held at 50 to 52° C. for 3 to 4 hours with constant stirring until the reaction was complete. To produce a smooth solution or if gelling of the product occurred, the reaction mixture was diluted to four times its volume with tetrahydrofuran or N-methylpyrrolidone. A 50/50 w/w mixture of methanol/water was added drop wise with rapid stirring in an amount sufficient to decompose the remaining anhydride(s) but insufficient to cause precipitation of the cellulose ester product. The solution was then cooled to ambient temperature creating a viscous smooth mixture referred to as “dope”. To separate the LCCE product, the dope was transferred to a separator funnel. To one part dope, 9 parts heptane/methylene chloride (9/1 w/w) was added and mixed with the dope. Then methanol was added and mixed with the dope in small portions until phase separation occurred. The mixture was allowed to rest between methanol additions. The liquid rich phase was drained away. Methanol addition and separation of the phases was repeated until the addition of a small portion of methanol began to precipitate the product. The resulting dope from this extraction process was stirred into excess methanol to precipitate the product. The product was separated from the precipitation liquids by filtration, washed with methanol several times, and then dried under vacuum and nitrogen at 50° C. The molecular weight and thermal transitions of the resulting cellulose esters were determined and are shown in Table 2.

TABLE 1

| Exam- | | | | Long Chain | |
|---|---|---|---|---|---|
| ple | | Cellu- | | Carboxylic | Acetic |
| Numb- | | lose | TFAA | Acid | Acid |
| er | EsterType | (grams) | (grams) | (grams) | (grams) |
| 10 | cellulose nonanoate | 15 | 134 | 137 | 0 |
| 11 | cellulose nonanoate | 15 | 134 | 137 | 0 |
| 12 | cellulose nonanoate | 15 | 134 | 137 | 0 |
| 13 | cellulose acetate | 14 | 125 | 104 | 6 |
| | nonanoate | | | | |
| 14 | cellulose acetate | 14 | 125 | 104 | 6 |
| | nonanoate | | | | |
| 15 | cellulose acetate | 14 | 125 | 104 | 6 |
| | nonanoate | | | | |
| 16 | cellulose isostearate | 15 | 134 | 200 | 0 |
| 17 | cellulose isostearate | 20 | 179 | 267 | 0 |
| 18 | cellulose isostearate | 20 | 179 | 267 | 0 |
| 19 | cellulose acetate | 20 | 179 | 231 | 8 |
| | isostearate | | | | |
| 20 | cellulose stearate | 10 | 97 | 162 | 0 |
| 21 | cellulose stearate | 10 | 89 | 133 | 0 |
| 22 | cellulose stearate | 18 | 161 | 240 | 0 |
| 23 | cellulose acetate | 23 | 181 | 204 | 9 |
| | stearate | | | | |

TABLE 2

| | | | Thermal |
|---|---|---|---|
| | | Weight-Average | Transition(s) by |
| | | Molecular Weight | Differential |
| | | in Tetrahydrofuran | Scanning |
| Example | | by GPC | Calorimetry |
| Number | EsterType | Mw | Approx. ° C. |
| 10 | cellulose nonanoate | 6.1 × 10⁵a | 90^{a} |
| 11 | cellulose nonanoate | | |
| 12 | cellulose nonanoate | | |
| 13 | cellulose acetate | 7.0 × 10⁵a | 84^{a} |
| | nonanoate | | |
| 14 | cellulose acetate | | |
| | nonanoate | | |
| 15 | cellulose acetate | | |
| | nonanoate | | |
| 16 | cellulose isostearate | 8.4 × 10⁵ | 110 |
| 17 | cellulose isostearate | 8.0 × 10⁶ | 98 |
| 18 | cellulose isostearate | not tested | Not tested |
| 19 | cellulose acetate | 4.8 × 10⁶ | 89 |
| | isostearate | | |
| 20 | cellulose stearate | 5.6 × 10⁶ | 32 (peak), 104 |
| 21 | cellulose stearate | 6.1 × 10⁶ | 35 (peak), 100 |
| 22 | cellulose stearate | 7.8 × 10⁶ | 51 (peak), 98 |
| 23 | cellulose acetate | 6.3 × 10⁶ | 26 (peak) |
| | stearate | | |

^{a}Samples from 3 examples were combined for test

Table 3 gives descriptions of mixtures resulting from mixing a long-chain cellulose ester (cellulose nonanoate combined samples from Examples 10-12) of the present invention with the specified cosmetically acceptable solvent at concentrations of 1, 2, and 4 weight %. For each mixture, the solvent and LCCE were weighed into a small vial. The vial was capped, and rolled overnight at about 65° C. The mixtures were observed after sitting at room temperature for 1 to 3 months.

TABLE 3

| | Cellulose Nonanoate | | |
|---|---|---|---|
| Solvent | 1% | 2% | 4% |
| isohexadecane | cl lo-vis liq | cl lo-vis liq | cl hi-vis |
| | | | liq |
| isododecane | cl lo-vis liq | cl lo-vis liq | cl med-vis |
| | | | liq |
| mineral oil (Drakeol 9) | cl lo-vis liq | cl med-vis liq | clear gel |
| ethylhexyl palmitate | cl lo-vis liq | cl lo-vis liq | cl hi-vis |
| | | | liq |
| C12-15 alkyl benzoate | not tested | not tested | cl med-vis |
| | | | liq |
| corn oil | cl lo-vis liq | cl med-vis liq | cl hi-vis |
| | | | liq |
| isocetyl alc (Jarcol I-16) | hazy liquid | not tested | not tested |
| isostearyl alc (Jarcol I-18T) | hazy liquid | not tested | not tested |
| isostearyl alc (Jarcol I-18EX) | cl lo-vis liq | cl lo-vis liq | cl med-vis |
| | | | liq |
| octyldodecanol (Jarcol I-20) | hazy liquid | not tested | not tested |
| isopalmitic acid (Jaric I-16) | insoluble | not tested | not tested |
| isostearic acid (Jaric I-18CG) | cl lo-vis liq | cl med-vis liq | cl hi-vis |
| | | | liq |
| oleic acid (Pamolyn 100) | cl lo-vis liq | cl lo-vis liq | cl med-vis |
| | | | liq |
| dimethicone (DC 200) | not tested | not tested | insoluble |
| cyclopentasiloxane (DC 245) | insoluble | not tested | not tested |
| 1/1 DC 245/isododecane | not tested | not tested | cl hi-vis |
| | | | liq |

Table 4 gives descriptions of mixtures resulting from mixing a long-chain cellulose ester (cellulose acetate nonanoate combined samples from Ex. 13-15) of the present invention with the specified cosmetically acceptable solvent at concentrations of 1, 2, and 4 weight %. For each mixture, the solvent and LCCE were weighed into a small vial. The vial was capped, and rolled overnight at about 65° C. The cellulose acetate nonanoate had a DS LCCE of 2.6 and a DS acetate of 0.42. The mixtures were observed after sitting at room temperature for 1 to 3 months.

TABLE 4

| | Cellulose Acetate Nonanoate | | |
|---|---|---|---|
| Solvent | 1% | 2% | 4% |
| isohexadecane | insoluble | clear gel | not tested |
| isododecane | cl lo-vis liq | clear gel | clear gel |
| mineral oil (Drakeol 9) | insoluble* | not tested | not tested |
| ethylhexyl palmitate | cl lo-vis liq | cl hi-vis liq | clear gel |
| C12-15 alkyl benzoate | not tested | not tested | cl med-vis |
| | | | liq |
| corn oil | cl med-vis | cl med-vis | cl hi-vis liq |
| | liq | liq | |
| isocetyl alc (Jarcol I-16) | cl lo-vis liq | cl hi-vis liq | hazy gel |
| isostearyl alc (Jarcol I-18T) | insoluble | not tested | not tested |
| isostearyl alc (Jarcol I-18EX) | cl lo-vis liq | clear gel | not tested |
| octyldodecanol (Jarcol I-20) | hazy liquid | not tested | not tested |
| isopalmitic acid (Jaric I-16) | cl lo-vis liq | cl hi-vis liq | clear gel |
| isostearic acid (Jaric I-18CG) | cl lo-vis liq | cl med-vis | cl hi vis liq |
| | | liq | |
| oleic acid (Pamolyn 100) | cl lo-vis liq | cl lo-vis liq | cl hi-vis liq |
| dimethicone (DC 200) | not tested | not tested | insoluble |
| cyclopentasiloxane (DC 245) | insoluble | not tested | not tested |
| 1/1 DC 245/isododecane | not tested | not tested | insoluble |

gel layer on bottom

Table 5 gives descriptions of mixtures resulting from mixing a long-chain cellulose ester (cellulose isostearate combined samples from Ex. 16 & 17) of the present invention with the specified cosmetically acceptable solvent at concentrations of 1, 2, and 4 weight %. For each mixture, the solvent and LCCE were weighed into a small vial. The vial was capped, and rolled overnight at about 65° C. The mixtures were observed after sitting at room temperature for 1 to 3 months.

TABLE 5

| | Cellulose Isostearate | | |
|---|---|---|---|
| Solvent | 1% | 2% | 4% |
| isohexadecane | cl lo-vis liq | cl lo-vis liq | clear gel |
| isododecane | cl lo-vis liq | cl lo-vis liq | cl hi vis |
| | | | liq |
| mineral oil (Drakeol 9) | insolube | not tested | not tested |
| ethylhexyl palmitate | cl lo-vis liq | cl lo-vis liq | cl med-vis |
| | | | liq |
| C12-15 alkyl benzoate | not tested | not tested | cl med-vis |
| | | | liq |
| corn oil | cl lo vis liq | cl lo vis liq | insoluble* |
| isocetyl alc (Jarcol I-16) | cl lo-vis liq | cl med-vis liq | cl hi-vis |
| | | | liq |
| isostearyl alc (Jarcol I-18T) | cl lo-vis liq | cl med-vis liq | cl hi-vis |
| | | | liq |
| isostearyl alc (Jarcol I-18EX) | cl lo-vis liq | cl med-vis liq | insoluble* |
| octyldodecanol (Jarcol I-20) | cl lo-vis liq | cl lomed-vis liq | cl hi-vis |
| | | | liq |
| isopalmitic acid (Jaric I-16) | cl lo-vis liq | cl hi-vis liq | clear gel |
| isostearic acid (Jaric I-18CG) | cl lo-vis liq | cl med-vis liq | insoluble* |
| oleic acid (Pamolyn 100) | cl lo-vis liq | cl lo-vis liq | insoluble |
| dimethicone (DC 200) | not tested | not tested | insoluble |
| cyclopentasiloxane (DC 245) | insoluble | not tested | not tested |
| 1/1 DC 245/isododecane | not tested | not tested | clear gel |

*Gel layer on bottom

Table 6 gives descriptions of mixtures resulting from mixing a long-chain cellulose ester (cellulose acetate isostearate sample from Example 19) of the present invention with the specified cosmetically acceptable solvent at concentrations of 1, 2, and 4 weight %. For each mixture, the solvent and LCCE were weighed into a small vial. The vial was capped, and rolled overnight at about 65° C. The mixtures were observed after sitting at room temperature for 1 to 3 months.

TABLE 6

| | Cellulose Acetate Isostearate | | |
|---|---|---|---|
| Solvent | 1% | 2% | 4% |
| isohexadecane | insoluble | not tested | not tested |
| isododecane | insoluble | not tested | not tested |
| mineral oil (Drakeol 9) | insoluble | not tested | not tested |
| ethylhexyl palmitate | insoluble | clear gel | not tested |
| C12-15 alkyl benzoate | not tested | not tested | clear gel |
| corn oil | insoluble | insoluble | not tested |
| isocetyl alc (Jarcol I-16) | insoluble | not tested | not tested |
| isostearyl alc (Jarcol I-18T) | insoluble | not tested | not tested |
| isostearyl alc (Jarcol I-18EX) | insoluble | not tested | not tested |
| octyldodecanol (Jarcol I-20) | insoluble | not tested | not tested |
| isopalmitic acid (Jaric I-16) | insoluble | not tested | not tested |
| isostearic acid (Jaric I-18CG) | insoluble | insoluble | insoluble |
| oleic acid (Pamolyn 100) | insoluble | clear gel | not tested |
| dimethicone (DC 200) | not tested | not tested | not tested |
| cyclopentasiloxane (DC 245) | insoluble | not tested | not tested |
| 1/1 DC 245/isododecane | not tested | not tested | not tested |

Table 7 gives descriptions of mixtures resulting from mixing a long-chain cellulose ester (cellulose stearate combined samples from Ex. 20-22) of the present invention with the specified cosmetically acceptable solvent at concentrations of 1 and 4 weight %. For each mixture, the solvent and LCCE were weighed into a small vial. The vial was capped, and rolled overnight at about 65° C. The mixtures were observed after sitting at room temperature for 1 to 3 months.

TABLE 7

Cellulose Stearate

| Solvent | 1% | 4% |
|---|---|---|
| isohexadecane | insoluble | not tested |
| isododecane | insoluble | not tested |
| mineral oil (Drakeol 9) | insoluble | not tested |
| ethylhexyl palmitate | hazy liq | not tested |
| C12-15 alkyl benzoate | not tested | hazy gel |
| corn oil | insoluble | not tested |
| isocetyl alc (Jarcol I-16) | insoluble | not tested |
| isostearyl alc (Jarcol I-18T) | insoluble | not tested |
| isostearyl alc (Jarcol I-18EX) | insoluble | not tested |
| octyldodecanol (Jarcol I-20) | insoluble | not tested |
| isopalmitic acid (Jaric I-16) | insoluble | not tested |
| isostearic acid (Jaric I-18CG) | insoluble | not tested |
| oleic acid (Pamolyn 100) | insoluble | not tested |
| dimethicone (DC 200) | not tested | not tested |
| cyclopentasiloxane (DC 245) | insoluble | not tested |
| 1/1 DC 245/isododecane | not tested | not tested |

Table 8 gives descriptions of mixtures resulting from mixing a long-chain cellulose ester (cellulose acetate stearate sample from Example 23) of the present invention with the specified cosmetically acceptable solvent at concentrations of 1, 2 and 4 weight %. For each mixture, the solvent and LCCE were weighed into a small vial. The vial was capped, and rolled overnight at about 65° C. The mixtures were observed after sitting at room temperature for 1 to 3 months.

TABLE 8

| | Cellulose Acetate Stearate | | |
|---|---|---|---|
| Solvent | 1% | 2% | 4% |
| isohexadecane | insoluble | not tested | not tested |
| isododecane | insoluble | not tested | not tested |
| mineral oil (Drakeol 9) | insoluble | not tested | not tested |
| ethylhexyl palmitate | insoluble | not tested | not tested |
| C12-15 alkyl benzoate | not tested | not tested | clear gel |
| corn oil | insoluble | not tested | not tested |
| isocetyl alc (Jarcol I-16) | insoluble | not tested | not tested |
| isostearyl alc (Jarcol I-18T) | insoluble | not tested | not tested |
| isostearyl alc (Jarcol I-18EX) | insoluble | not tested | not tested |
| octyldodecanol (Jarcol I-20) | insoluble | not tested | not tested |
| isopalmitic acid (Jaric I-16) | insoluble | not tested | not tested |
| isostearic acid (Jaric I-18CG) | insoluble | clear gel | clear gel |
| oleic acid (Pamolyn 100) | insoluble | clear gel | not tested |
| dimethicone (DC 200) | not tested | not tested | not tested |
| cyclopentasiloxane (DC 245) | insoluble | not tested | not tested |
| 1/1 DC 245/isododecane | not tested- | not tested | not tested |

In Tables 3-8 above, viscosity was determined using a shear rate of 1 to 5 rad/sec with low viscosity being defined as less than 500 centipoise; medium viscosity being defined as between 500 and 2000 centipoise; and high viscosity being defined as greater than 2000 centipoise.

### COMPARATIVE EXAMPLE 24

Preparation of Isostearoyl Chloride:

Isostearic acid (80.1 grams, 0.28 moles, available from A & E Connock) was added to a round bottom flask, equipped with a condenser type distilling head, mechanical stirrer, and a thermostatically-controlled oil bath. The initial temperature of the 2 liter oil was about 25° C. Over a time period of 50 minutes, thionyl chloride (39 grams, 0.33 moles, available from Aldrich Chemical Company) was added drop-wise to the isostearic acid, with constant stirring. At about halfway through the addition the oil bath temperature was raised to 35° C. and the reaction was stirred for an additional 2 hours. Vacuum was applied (90 mm Hg) to the distillation column and the oil bath temperature was increased to 50° C. Unreacted thionyl chloride (4.5 grams) was distilled away from the product yielding 85 grams of isostearoyl chloride, which was used without further purification.

A specimen of cellulose acetate butyrate having lateral isostearyl ester groups was attempted to be prepared following the procedure described in Example 1 of the PCT International patent publication WO 2005/013926. The batch size was 25% of that disclosed in Example 1. The reagents were added, in the following order to a room temperature, 1000 mL, three-neck, round bottom flask equipped with a stirrer, cold water cooled condenser, vented to a drying tube filled with anhydrous calcium sulfate, a dry nitrogen inlet tube, and placed in a silicone oil bath:

225 grams of toluene (Burdick and Jackson—B&J High Purity Solvent grade); and

225 grams of methyl ethyl ketone (Mallinckrodt—analytical reagent grade).

Slowly with rapid stirring, 25 grams of cellulose acetate butyrate (EASTMAN CAB-553-0.4-46.43 weight % butyryl) was added and dissolved by heating to 50° C. with continual stirring for 1 hour. The mixture was cooled to room temperature and 5.0 grams of triethylamine (Aldrich) is added to the mixture. With continual stirring and under a dry nitrogen atmosphere the flask was cooled in an ice bath to +5° C. When the mixture reached a temperature of +5° C., 14.22 grams of the isostearoyl chloride prepared above (dissolved in 25 grams of toluene and 25 grams of methyl ethyl ketone) was added dropwise from an addition funnel over 1 hour and 30 minutes. The temperature was measured several times during addition with a hand held electronic thermometer. Maximum temperature reached in the reaction mixture was 7.1° C. The reaction mixture was removed from the ice bath and returned to room temperature (22° C.) and held with constant slow stirring for 18 hours. Crystals believed to be triethylamine hydrochloride clouded the reaction mixture but were not seen on the sides of the flask.

The resulting mixture was filtered with a medium fitted glass funnel and then through filter paper. Portions of the reaction mixture were individually stirred into methanol, ethanol and isopropanol. No filterable precipitate formed only a hazy yellow solution or milky solution formed with no filterable precipitate. The balance of the reaction mixture was stirred into methanol/water, 50/50, w/w, forming a milky solution and a greasy yellow liquid precipitate phase slowly formed. The precipitate was believed to be isostearic acid resulting from the decomposition of unreacted isostearoyl chloride with water in the precipitation liquid and no sample was isolated. No measurable cellulose acetate butyrate isostearate was recovered using this process.

### EXAMPLE 24A

Specimens of cellulose acetate butyrate isostearate were prepared by the procedure described in Example 1 of the PCT International Patent Application WO2005/013926, except the product was precipitated in heptane instead of alcohol. Two batches were prepared; each batch size was 25% of that disclosed in Example 1 of the International Patent Application WO2005/013926 patent publication. The reagents were added, in the following order to each of two 1000 mL, three-neck, round bottom flasks, equipped with stirrers, cold water cooled condensers vented to drying tubes filled with anhydrous calcium sulfate, and dry nitrogen inlets, and placed in silicone oil baths:

225 grams of toluene per flask—Burdick and Jackson—B&J High Purity Solvent grade

225 grams of methyl ethyl ketone per flask—Mallinckrodt—analytical reagent grade

With rapid stirring, 25 grams of cellulose acetate butyrate (EASTMAN CAB-553-0.4-46.43 weight % butyryl) was slowly added to each flask and dissolved by heating to 50° C. with continual stirring for 1 hour. The mixtures were cooled to room temperature and 7.5 grams of triethylamine (Aldrich) was added to each flask (excess triethylamine to prevent HCL degradation of the product ester). With continual stirring and under a dry nitrogen atmosphere the flasks were cooled in an ice bath to +5° C.

When the mixtures reached +5° C., 20.00 grams of the isostearoyl chloride prepared above (dissolved in 25 grams of toluene and 25 grams of methyl ethyl ketone) was added dropwise from addition funnels to each flask over approximately 1 hour and 30 minutes. The temperature of the flasks was measured several times during addition using a hand held electronic thermometer. Maximum temperature reached in the reaction mixtures was 7.6° C. The reaction mixtures were removed from the ice bath and returned to room temperature (22° C.) and held with constant slow stirring for 18 hours. Crystals believed to be triethylamine hydrochloride clouded the reaction mixtures but were not seen on the sides of the flasks.

The resulting mixtures from both flasks were combined, filtered using a medium fritted glass funnel, then through filter paper, and precipitated in heptane. The mixture made a white flaky precipitate that was large and solid enough to filter from the precipitation liquids. The precipitate was washed twice in heptane and dried to a constant weight under nitrogen and vacuum at 55° C. The recovered precipitate weighed 46.96 grams and had a DS for isostearate of 0.17. The product had a weight-average molecular weight of 3.2×10⁴ as measured by gel permeation chromatography. The product was insoluble in isododecane and isohexadecane.

### EXAMPLES 25-27

Various solvents were investigated to determine which would solubilize cellulose nonanoate (CN), cellulose acetate nonanoate (CAN), and cellulose isostearate (CIS) at the highest concentration, with the intention of making drawdowns of the solutions and drying to produce films. Solvents tested were methyl acetate, butyl acetate, n-methyl pyrrolidone, mineral spirits, cyclohexanone, isophorone, methanol, and Aromatic 100 hydrocarbon fluid (ExxonMobil). Aromatic 100 hydrocarbon fluid was found to be most suitable, solubilizing all three LCCEs at 15 weight %. The other solvents would not solubilize the LCCEs at concentrations greater than 10 weight %. Solutions of the specified LCCEs were made at 15 weight % in Aromatic 100 fluid. Drawdowns of the solutions were made on to metal plates and Leneta chart paper. The films were dried at room temperature. The resulting films were clear having an average thickness of about 2 mils. The films were tested for Pendulum hardness (ASTM D4366-87), Tukon hardness (ASTM D1474), gloss, and flexibility by a flex bar test and tactile evaluation. Results are given in Table 9 below. Each test result is the average of 3 measurements.

TABLE 9

| | CN | CAN | CIS |
|---|---|---|---|
| Pendulum Hardness, | 42 | 62 | 8 |
| sec | | | |
| Tukon Hardness, | 1.0 | 1.5 | <0.5 |
| knoops | | | |
| Flex Bar Test | No break thru | No break thru | No break thru |
| | ⅛ inch | ⅛ inch | ⅛ inch |
| Flexibility Ranking* | 2 | 3 | 1 |
| Gloss Units | 80 | 83 | 83 |
| Refractive Index | 1.473 | 1.472 | 1.476 |

*1 is the softest and most flexible, 3 is the hardest and least flexible. All three films had a good gloss, and can contribute gloss to a personal care product if desired.

### EXAMPLES 28-31

In the following examples, a personal care product, (mascara) was prepared using a long chain fatty acid cellulose ester as described above that is soluble in a suitably cosmetically acceptable solvent. The specified amounts of wax, stearic acid, ethylhexyl palmitate, and the LCCE were weighed into a beaker and heated to 80° C. The ingredients were mixed when melted to obtain a homogeneous mixture. The gum arabic was added to the water and allowed to hydrolyze overnight at room temperature. The water/gum mixture was heated to 50° C. with stirring while slowly adding the hydroxyethylcellulose, followed by triethanolamine. The aqueous phase was heated to 80° C.; then the wax phase was slowly added to the aqueous phase while mixing. The mixture was cooled to 40° C. and the preservative was added. Mixing was continued until blended. The results are presented in Table 10 below.

TABLE 10

| | | | Com- | | | | | |
|---|---|---|---|---|---|---|---|---|
| | Example | | parative | | Comparative | | Example | |
| Ingredient | 28 | | Ex. 29 | | Ex. 30 | | 31 | |
| Candelilla wax | 4.9 | g | 4.9 | g | 4.9 | g | 3.1 | g |
| Paraffin wax | 4.3 | | 4.3 | | 4.3 | | 2.7 | |
| Beeswax | 9.2 | | 9.2 | | 9.2 | | 5.8 | |
| Stearic acid | 6.3 | | 6.3 | | 6.3 | | 4.0 | |
| Ethylhexyl palmitate | 10.8 | | 10.8 | | 10.8 | | 6.8 | |

| | | | | |
|---|---|---|---|---|
| CN | 5.4 | — | — | — |

| | | | | |
|---|---|---|---|---|
| CAN | — | 5.4 | — | — |

| CIS | — | — | 5.4 | 3.4 |
|---|---|---|---|---|
| Water | 51.5 | — | 51.5 | 64.7 |
| Gum Arabic | 3.7 | — | 3.7 | 4.6 |
| Hydroxyethylcellulose | 1.0 | — | 1.0 | 1.3 |
| Triethanolamine | 2.6 | — | 2.6 | 3.3 |
| Preservative | 0.3 | — | 0.3 | 0.3 |
| (Germaben II) | | | | |

The example with CN had a creamy consistency. When applied to eyelashes, it separates and defines the lashes. The formulation given as the comparative example with CAN was not completed because the CAN did not dissolve in the melted wax phase ingredients. The first example with CIS formed a solid, not suitable for use as a mascara. The second formulation with CIS with a reduced wax phase concentration has a creamy consistency. When applied to eyelashes, it separates and defines the lashes. The formulation of this example was also applied and spread on the skin. It spread easily, felt smooth as it was spread, and left a water-resistant film on the skin. As is known in the art, pigments may be added to the above formulations.

### EXAMPLE 32

In the following example, a personal care product, (lipstick) was prepared using a long chain fatty acid cellulose ester as described above that is soluble in a suitably cosmetically acceptable solvent. The ingredients were weighed into a jar and placed in an oven at 95° C. When all ingredients had melted, they were mixed until homogeneous. As the mixture cooled it was poured into a lipstick mold. The resulting lipstick was evaluated by applying to the skin. The specified amounts and the ingredients are presented in Table 11 below.

TABLE 11

| Ingredient: | Weight % |
|---|---|
| Polyethylene wax | 30.8 |
| CIS | 4.7 |
| Isododecane | 46.1 |
| Hydrogenated polyisobutene | 7.7 |
| Isostearyl alcohol | 2.8 |
| Phenyl trimethicone | 3.6 |
| C30-39 olefin/isopropylmaleate/MA | 0.9 |
| Copolymer | |
| Pigment Wax Dispersion | 3.2 |

The stick had poor glide, but deposited the color well. A few minutes after application, it felt dry and was not greasy. Color adhered well; would not rub off.

### EXAMPLES 33-35

In the following examples, hair styling products were prepared. Hair tresses were prepared by combing, wetting, and removing excess water. An equal amount (0.2 g) of each solution or gel of 4% CN, CAN and CIS in isododecane were applied to hair tresses weighing about 2.8 g by working the solution or gel through the hair with the fingers. The tresses were combed after applying the solution or gel and allowed to air dry overnight. After drying, a curling iron was used to curl the hair tresses. Compared to the untreated hair tress, the LCCE treated tresses were easier to comb, had more shine, and better curl retention under high humidity conditions. CN and CIS provided more gloss and better hold compared to CAN. CIS provided a flexible hold, while CN provided a stiffer hold. After application the tresses were washed with shampoo. The LCCEs were difficult to wash from the hair. Remaining LCCE could be seen on the hair as white specks after the hair had dried.

As indicated by the poor wash-out, it is apparent that the LCCEs have good substantivity to the hair and therefore have utility as temporary hair dyes. A hair dye is incorporated into the solution of LCCE in isododecane and applied to the hair.

To produce a low-VOC product that meets low-VOC regulations, isohexadecane was used in place of isododecane. The tresses required more time to dry and retained their oily feel after a few hours at room temperature. However, treatment with heat, for example with a hair drier or curling iron, quickly removed the isohexadecane solvent leaving behind a glossy finish, good manageability, and curl retention.

### EXAMPLE 36

A sun protection product was prepared having the composition specified in Table 12 below.

TABLE 12

| Ingredient | Weight % |
|---|---|
| C12-15 alkyl benzoate (Finsolv TN) | 5.0 |
| Ethylhexyl methoxycinnamate | 7.0 |
| Butyloctyl salicylate | 2.0 |
| Octocrylene | 2.0 |
| Benzophenone-3 | 4.0 |
| Butyl methoxydibenzoylmethane | 2.0 |
| Cetearyl alcohol (and) dicetyl phosphate | 2.0 |
| (and) ceteth-10 phosphate | |
| Myristyl myristate | 3.0 |
| Glyceryl stearate | 1.0 |
| Cellulose Nonanoate | 1.0 |
| Polyester-5 | 3.0 |
| Water | 68.0 |
| Triethanolamine | 0.1 |

The oil phase ingredients and water phase ingredients were mixed separately at 80° C., then combined and mixed with a high shear mixer for 10 minutes. The resulting low-viscosity emulsion had a smooth feel when applied to the skin and left behind a water-resistant film.

### EXAMPLES 37 & 38

An antiperspirant product was prepared having the composition (weight %) specified in Table 13 below. The ingredients, except for the aluminum/zirconium tetrachlorohydrex-Gly (AAZG-7167, Summit Research Lab), were weighed into a beaker and heated with stirring to 85° C. When the mixture appeared homogeneous, the tetrachlorohydrex-Gly was added and dispersed using a high-speed disperser. The formulation was mixed for 10 minutes at 82° C., then allowed to cool. When the temperature reached 60° C., the mixture was poured into an antiperspirant stick container. After sitting overnight at room temperature the antiperspirant sticks were evaluated. Both set up to form white opaque sticks. When applied to the skin, both have a smooth feel.

TABLE 13

| | | Comparative |
|---|---|---|
| Ingredient | Example 37 | Example 38 |
| Cetearyl alcohol | 20.0 | 20.0 |
| Cyclopentasiloxane | 26.5 | 30.0 |
| Isododecane | 26.0 | 26.0 |
| CIS | 3.5 | — |
| Aluminum/zirconium | 24.0 | 24.0 |
| tetrachlorohydrex-Gly | | |

The formulation with CIS was observed to deposit more material on the skin. After drying the CIS formulation provided a more comfortable feel on the skin, with no sensation of skin tightening. With very hard rubbing, the sample with CIS rolled up off of the skin indicating a film had been left behind, whereas the sample without CIS did not have this effect.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents. Moreover, all patents, patent applications, publications, and literature references presented herein are incorporated by reference in their entirety for any disclosure pertinent to the practice of this invention.

We claim:

1. A personal care product composition comprising a long chain fatty acid cellulose ester (LCCE) having a degree of substitution greater than about 1.0 of an ester substituent or residue from fatty acids having from 6 to 18 carbon atoms wherein the LCCE is soluble in at least one cosmetically acceptable solvent selected from the group consisting of hydrocarbons, alkyl esters, fats and oils, fatty acids, fatty alcohols, and silicone oils.

2. The personal care product composition of claim 1 wherein the amount of long chain fatty acid cellulose ester is from about 0.1 to about 50 weight %, based on the total weight of constituents present in said composition.

3. The personal care product composition of claim 1 wherein the amount of long chain fatty acid cellulose ester is from about 0.5 to about 30 weight %, based on the total weight of constituents present in said composition.

4. The personal care product composition of claim 1 wherein the amount of long chain fatty acid cellulose ester is from about 0.5 to about 15 weight %, based on the total weight of constituents present in said composition.

5. The personal care product composition of claim 1 wherein said long chain fatty acid cellulose ester has a degree of substitution greater than about 1.5 of an ester substituent or residue from fatty acids having from 8 to 18 carbon atoms.

6. The personal care product composition of claim 1 wherein said long chain fatty acid cellulose ester has a degree of substitution greater than about 2.0 of an ester substituent or residue from fatty acids having from 9 to 18 carbon atoms.

7. The personal care product composition of claim 1 wherein said long chain fatty acid cellulose ester has a degree of substitution greater than about 2.5 of an ester substituent or residue from fatty acids having from 9 to 18 carbon atoms.

8. The personal care product composition of any one of claims 1, 5, 6 or 7 wherein said long chain fatty acid cellulose ester has an acetyl degree of substitution of less than about 0.5.

9. The personal care product composition of claim 8 wherein said long chain fatty acid cellulose ester has an acetyl degree of substitution of less than about 0.3.

10. The personal care product composition of claim 1 wherein said long chain fatty acid cellulose ester is selected from the group consisting of cellulose palmitate, cellulose nonanoate, cellulose isostearate, cellulose hexanoate, cellulose acetate hexanoate, cellulose acetate nonanoate, cellulose acetate laurate, cellulose acetate stearate, cellulose hexanoate propionate, and cellulose nonanoate propionate.

11. The personal care product composition of claim 1 wherein said cosmetically acceptable hydrocarbon solvent is selected from the group consisting of isoparaffins, hydrogenated polyisobutene, isododecane, isoeicosane, isohexadecane, isopentane, microcrystalline wax, mineral oil, mineral spirits, paraffin, petrolatum, squalene, polyethylene, camauba wax, candelilla wax and mixtures thereof.

12. The personal care product composition of claim 1 wherein said cosmetically acceptable alkyl ester solvent is selected from the group consisting of alkyl acetates, alkyl behenates, alkyl lactates, alkyl benzoates, alkyl salicylates, typical alkyl fatty acid esters such as alkyl stearates, alkyl myristates, alky laurates, and mixtures thereof, wherein the alkyl portion has at least eight carbon atoms.

13. The personal care product composition of claim 1 wherein said cosmetically acceptable fats and oils are selected from the group consisting of soybean oil, canola oil, olive oil, sunflower oil, triolein, tristearin, caprylic/capric triglyceride, and mixtures thereof.

14. The personal care product composition of claim 1 wherein said cosmetically acceptable fatty acid is selected from the group consisting of valeric acid, heptylic acid, caprylic acid, lauric acid, myristic acid, and palmitic acid behenic acid, capric acid, caproic acid, coconut acid, oleic acid, linoleic acid, palmitic acid, stearic acid, and mixture thereof.

15. The personal care product composition of claim 1 wherein said cosmetically acceptable fatty alcohols are selected from C₉-C₁₈ alcohols.

16. The personal care product composition of claim 1 wherein said cosmetically acceptable silicone oil is selected from the group consisting of dimethicone, hexadecyl methicone, stearyl dimethicone, and mixtures thereof.

17. The personal care product composition of claim 1 wherein said personal care product is selected from the group consisting of deodorants, antiperspirants, combination antiperspirant deodorants, shaving products, skin lotions, moisturizers, toners, bath products, cleansing products, hair care products, shampoos, conditioners, mousses, styling gels, hair sprays, hair dyes, hair coloring products, hair bleaches, hair waving products, hair straighteners, manicure products, nail polish, nail polish remover, nail creams, nail lotions, cuticle softeners, protective creams, sunscreen products, insect repellent, anti-aging products, color cosmetics, lipsticks, foundations, face powders, eye liners, eye shadows, blushes, makeup, and mascara.

18. The personal care product composition of claim 1 further comprising components selected from the group consisting of cleansing agents, emollients, moisturizers, pigments, colorants, fragrances, biocides, preservatives, antioxidants, antiperspirant agents, oral care agents, exfoliants, hormones, enzymes, medicinal compounds, vitamins, ultraviolet light absorbers, dihydroxyacetone, skin bleaching agents, antiacne agents, botanical extracts, silicone oils, organic oils, waxes, adhesion promoters, plasticizers, film formers, including hair fixatives, thickening agents, fillers and binders, alcohol and propellants.

19. A personal care product composition containing a long chain fatty acid cellulose ester (LCCE) having a degree of substitution greater than about 1.0 of an ester substituent or residue from fatty acids having from 9 to 18 carbon atoms and a degree of substitution of an acetyl moiety of less than about 0.5, wherein the LCCE is soluble in at least one cosmetically acceptable solvent selected from the group consisting of isoparaffins, hydrogenated polyisobutene, isododecane, isoeicosane, isohexadecane, isopentane, mineral oil, mineral spirits, paraffin, petrolatum, squalene, and C₉-C₁₈ alcohols.
